(19) United States
(12) Patent Application Publication
(10) Pub. No.: US 2008/0214596 A1
(43) Pub. Date: Sep. 4, 2008

(54) BIOMARKERS FOR SENSITIVITY OF PROLIFERATIVE DISEASES TO MTOR INHIBITORS

(76) Inventors: Anne Boulay, Blotzheim (FR); Heidi Lane, Basel (CH); Sauveur-Michel Maira, Habsheim (FR); Terence O' Reilly, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/119,640

(22) Filed: May 13, 2008

Related U.S. Application Data
(63) Continuation of application No. 10/583,674, filed on Jul. 31, 2006, now abandoned.
(60) Provisional application No. 60/531,700, filed on Dec. 22, 2003.

(51) Int. Cl. A61K 31/436; G01N 33/68; A61P 35/00

(52) U.S. Cl. 514/291; 436/64

(57) ABSTRACT

Disclosed is a method for determining the sensitivity of a proliferate disease in a subject to treatment with an mTOR inhibitor, comprising determining the level of expression and/or phosphorylation state of S6 in a sample derived from the subject, as well as related methods of treatment and uses.

The present invention relates to biomarkers for determining the sensitivity of proliferative diseases such as cancer to therapeutic agents, in particular mTOR inhibitors.

A number of mTOR inhibitors have potent antiproliferative properties which make them useful for cancer chemotherapy, particularly of solid tumors, especially of advanced solid tumors. However there is still a need for more targeted use of mTOR inhibitors, which requires identification of patients which are likely to respond to treatment with such agents. Accordingly there is a need for biomarkers useful in e.g. clinical tests, which are capable of predicting responsiveness of a proliferative disease, e.g. a tumor in a patient to treatment with an mTOR inhibitor.

It has surprisingly been found that S6 40S ribosomal protein (otherwise known as S6) is a useful biomarker which is predictive of sensitivity of proliferative diseases to treatment with an mTOR inhibitor. In particular, it has been found that the phosphorylation state of S6 correlates well with sensitivity to mTOR inhibitors. mTOR inhibitors are more likely to show a significant antiproliferative effect when used to treat cancer cell lines showing higher levels of expression of phosphorylated S6. S6 is a component of the 40S ribosomal subunit which is a substrate for the p70 S6 kinase, a downstream effector of the mTOR protein kinase. Multiple phosphorylation of S6 has been implicated in the translational upregulation of mRNAs encoding components of the protein synthetic apparatus, and as such is thought to play a major role in the growth of mammalian cells (Volarevic and Thomas, Prog. Nucleic Acid Res. Mol. Biol. 2001, 65:101-27). The sequence of human S6 is available under Genbank accession number M20020.

The present invention provides in one aspect use of S6 40S ribosomal protein (S6), in particular phosphorylated S6, as a biomarker for determining the sensitivity of a proliferative disease to treatment with an mTOR inhibitor.

In a further aspect the invention provides a method for determining the sensitivity of a proliferative disease in a subject to treatment with an mTOR inhibitor, comprising determining the level of expression and/or phosphorylation state of S6 in a sample derived from the subject.

In another aspect the invention provides a method of selecting subjects suffering from a proliferative disease for treatment with an mTOR inhibitor, comprising determining the sensitivity of the proliferative disease to treatment with an mTOR inhibitor in each subject by a method as described above, and selecting those subjects showing increased expression of phosphorylated S6 for treatment with an mTOR inhibitor.

The term “mTOR Inhibitor” as used herein includes, but is not limited to rapamycin (sirolimus) or a derivative thereof. Rapamycin is a known macrolide antibiotic produced by Streptomyces hygroscopicus. Suitable derivatives of rapamycin include e.g. compounds of formula A

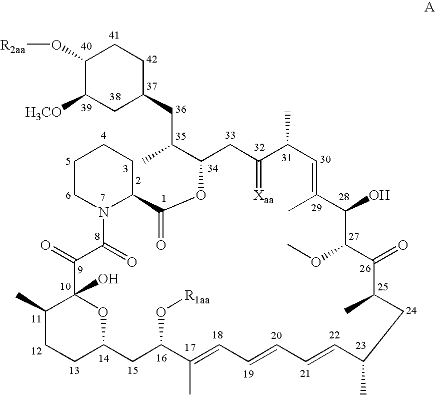

wherein
R₁ₐₐ is CH₃ or C₃₋₆alkynyl,
R2aa is H or —CH₂—CH₂—OH, 3-hydroxy-2-(hydroxymethyl)-2-methyl-propanoyl or tetrazolyl, and

### Xₐₐ is =0, (H,H) or (H,OH)

provided that R₂ₐₐ is other than H when Xₐₐ is =0 and R₁ₐₐ is CH₃.
or a prodrug thereof when R₂ₐₐ is —CH₂—CH₂—OH, e.g. a physiologically hydrolysable ether thereof, e.g. a compound wherein R₂ₐₐ is —CH₂—CH₂—O— Alk, Alk being a C₁₋₉alkyl optionally interrupted in the chain by 1 or 2 oxygen atoms.

Compounds of formula A are disclosed e.g. in WO 94/09010, WO 95/16691, WO 96/41807, U.S. Pat. No. 5,362,718 or WO 99/15530 which are incorporated herein by reference. They may be prepared as disclosed or by analogy to the procedures described in these references.

Preferred rapamycin derivatives are 32-deoxorapamycin, 16-pent-2-ynyloxy-32-deoxorapamycin, 16-pent-2-ynyloxy-32(S)-dihydro-rapamycin, 16-pent-2-ynyloxy-32(S) dihydro-40-O-(2-hydroxyethyl)-rapamycin and, more preferably, 40-O-(2-hydroxyethyl) rapamycin. Further examples of rapamycin derivatives include e.g. CCl779 or 40-[3-hydroxy-2-(hydroxymethyl)-2-methylpropanoate]-rapamycin or a pharmaceutically acceptable salt thereof, as disclosed in U.S. Pat. No. 5,362,718, ABT578 or 40-(tetrazolyl)-rapamycin, particularly 40epi-(tetrazolylrapamycin, e.g. as disclosed in WO 99/15530. Rapamycin derivatives may also include the so-called rapalogs, e.g. as disclosed in WO 98/02441, WO01/14387 and WO 03/64383, e.g. AP23573, AP23464, AP23675 or AP23841. Further examples of a rapamycin derivative are those disclosed under the name TAFA-93, biolimus-7 or biolimus-9.

In each case where citations of patent applications or scientific publications are given, the subject-matter relating to the compounds is hereby incorporated into the present application by reference. Comprised are likewise the pharmaceutically acceptable salts thereof, the corresponding racemates, diastereoisomers, enantiomers, tautomers as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively.

The proliferative disease may be a benign or malignant proliferaUve disease, e.g. benign prostatic hyperplasia, or a neoplastic disease, preferably a malignant proliferative disease, e.g. a cancer, e.g. a solid tumor, particularly an advanced solid tumor as disclosed in WO 02/66019. By “solid tumors” are meant tumors and/or metastasis (wherever located) other than lymphatic cancer, e.g. brain and other central nervous system tumors (eg. tumors of the meninges, brain, spinal cord, cranial nerves and other parts of central nervous system, e.g. glioblastomas or medulla blastomas); head and/or neck cancer; breast tumors; circulatory system tumors (e.g. heart, mediasUnum and pleura, and other inbrathoracic organs, vascular tumors and tumor-associated vascular tissue); excretory system tumors (e.g. kidney, renal pelvis, ureter, bladder, other and unspecified urinary organs); gastrointestinal tract tumors (e.g. oesophagus, stomach, small intestine, colon, colorectal, rectosigmoid junction, rectum, anus and anal canal), tumors involving the liver and intrahepatic bile ducts, gall bladder, other and unspecified parts of biliary tract, pancreas, other and digestive organs); head and neck; oral cavity (lip, tongue, gum, floor of mouth, palate, and other parts of mouth, paroud gland, and other parts of the salivary glands, tonsil, oropharynx, nasopharynx, pyriform sinus, hypopharynx, and other sites in the lip, oral cavity and pharynx); reproductive system tumors (e.g. vulva, vagina, Cervix uteri, Corpus uteri, uterus, ovary, and other sites associated with female genital organs, placenta, penis, prostate, tests, and other sites associated with male genital organs); respiratory tract tumors (e.g. nasal cavity and middle ear, accessory sinuses, larynx, trachea, bronchus and lung, e.g. small cell lung cancer or non-small cell lung cancer); skeletal system tumors (e.g. bone and articular cartilage of limbs, bone articular cartilage and other sites); skin tumors (e.g. malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites. Where hereinbefore and subsequently a tumor, a tumor disease, a carcinoma or a cancer is mentioned, also metastasis in the original organ or tissue and/or in any other location are implied alternatively or in addition, whatever the location of the tumor and/or metastasis is.

According to the method of the present invention, subjects suffering from such a proliferative disease can be screened in order to predict their sensitivity to mTOR inhibitors. The method may be performed in vitro, e.g. on a sample of biological tissue derived from the subject. The sample may be any biological material separated from the mammalian body such as e.g. tissue, cell lines, plasma or serum, cell or tissue lysate, preferably tumor tissue. The subject is preferably a human subject.

Levels of expression and/or phosphorylation state of S6 are assayed in the biological sample by any technical means on the basis of e.g. RNA expression using for example the technique of RT-PCR or on the basis of e.g. protein expression using for example the technique of Western blotting, immunohistochemistry or ELISA, including immunoassays, immunoprecipitation and electrophoresis assays. Preferably the method comprises determining the level of expression of (e.g. human) S6 protein, and in particular phosphorylated S6 in the sample. The method may involve detection of phosphorylation at any phosphorylation site on S6. For example, phosphorylation of (e.g. human) S6 on serine 235/236 may be determined, more preferably phosphorylation of S6 on serines 240/244 is determined.

For example, antibodies specific for (e.g. phosphorylated) S6 are used in a standard immunoassay format to measure (e.g. phosphorylated) S6 levels. ELISA (enzyme linked immunosorbent assay) type assays, immunoprecipitation type assays, conventional Western blotting assays and immunohistochemistry assays using e.g. monoclonal or polyclonal antibodies are also utilized to determine levels of the phosphorylated S6 as a biomarker protein.

Polyclonal and monoclonal antibodies specific to S6, e.g. to S6 protein or to phosphorylated S6 are produced in accordance with known immunization methods.

The phosphorylated S6 level may also be measured by two-dimensional (2-D) gel electrophoresis. 2-D gel electrophoresis is known in the art and typically involves isoelectric focusing (IEF) along a first dimension followed by SDS-PAGE (sodium dodecyl sulphate-polyacrylamide gel electrophoresis) along a second dimension. The resulting electropherograms are analyzed, for example, by immunoblot analysis using antibodies. Suitable antibodies directed against S6 protein or phosphorylated S6 can be produced as discussed above or obtained from a commercial source (e.g. Cell Signaling Technology® catalogue # 2212; #2215; #2211).

The present invention thus provides a method of screening subjects suffering from a proliferative disease in order to predict their responsiveness to treatment with an mTOR inhibitor, comprising determining the level of expression and/or phosphorylation state of S6 by a method as defined above.

In a further aspect, the present invention provides a method of treating a proliferative disease in a subject in need thereof, comprising determining the level of expression and/or phosphorylation state of S6 in a sample derived from the subject, by a method as described above, and treating the subject with an mTOR inhibitor if the level of expression of (e.g. phosphorylated) S6 is elevated.

The level found in a particular tissue from a subject, e.g. a sample of tumor tissue, may be compared with a control sample, e.g. a sample of normal tissue from a subject not suffering from the disease, or a sample of normal (i.e non-tumor) tissue from the same subject. An elevated level of phosphorylated S6, e.g. above control levels, is predictive of a beneficial therapeutic effect (i.e. an antiproliferative effect) of an mTOR inhibitor. The elevated level at which use of an mTOR inhibitor is indicated may be determined by a skilled person, e.g. in certain embodiments treatment with an mTOR inhibitor may be indicated where the level of phosphorylated S6 in the sample is detectably above the control level, or where the level is at least 50%, 100%, 500% or 1000% higher than control.

Moreover, the method may be used to select an appropriate dose of an mTOR inhibitor in order to individually optimise therapy for each patient. For instance a lower dose of an mTOR inhibitor may be selected where a sample from the subject shows higher phosphor-S6 levels, and vice versa. Factors for consideration in this context include the particular condition being treated, the particular mammal being treated, the clinical condition of the individual patient, the site of delivery of the active compound, the particular type of the active compound, the method of administration, the scheduling of administration, the severity of the condition and other factors known to medical practitioners. The therapeutically effective amount of an active compound to be administered will be governed by such considerations, and is the minimum amount necessary to prevent, ameliorate, or treat the disease. Such amount is preferably below the amount that is toxic to the host or which renders the host significantly more susceptible to infections. Appropriates doses of an mTOR inhibitor are e.g. as disclosed in WO 02/66019, e.g. daily dosage rates of the order of ca. 0.1 to 70 mg, e.g. from ca. 0.1 to 25 mg, for instance from ca. 0.05 to 10 mg active ingredient p.o., as a single dose or in divided doses or intermittent, e.g. once a week. Rapamycin or a derivative thereof, e.g. a compound of formula A, may be administered by any conventional route, in particular enterally, e.g. orally, e.g. in the form of tablets, capsules, drink solutions or parenterally, e.g. in the form of injectable solutions or suspensions, containing, for example, from about 0.1% to about 99.9%, preferably from about 1% to about 60%, of the active ingredient(s).

### EXAMPLE 1

Human tumor cell lines, e.g. the 40-O-(2-hydroxyethyl) rapamycin-sensitive MCF7, BT549 or LNCap lines (IC₅₀ in sub nM range) versus the comparative 40-O-(2-hydroxyethyl) rapamycin-resistant PC3M line (IC₅₀ In the >100 nM range), as well as cell lines with moderate rapamcyin-sensitivity (IC₅₀ in the 1 nM-100 nM range) such as DU145, HCC1937 and MDA-MB231, are added to 96-well plates (500 to 5000 cells/well in 100 μl medium) and incubated for 24 hr. Subsequently, a dilution series of an mTOR inhibitor, e.g. a compound of formula A, e.g. 40-O-(2-hydroxyethyl) rapamycin is made in separate wells and the dilutions are added to the wells. The cells are then reincubated for 4 days. Methylene blue staining is performed on day 5 and the amount of bound dye (proportional to the number of surviving cells that bind the dye) determined. IC₅₀s are subsequently determined using Softmax 1.2.0 software.

The same tumor cell lines as above, cultured to 50-70% confluency, are refed with normal culture medium (10% v/v FCS). After 24 hours, protein lysates are prepared and 20 μg electrophoretically resolved and transferred to polyvinylidene difluoride (PVDF) by semi-dry electroblotting. Blots are probed with anti-phospho-S6 or anti-S6 protein antibody and decorated proteins are revealed using enhanced chemiluminescence. The relative intensities of S6 phosphorylation in each cell line are revealed and numerated as: 0 (no phosphorylation observed), 0.5, 1, 2, 3 or 4 (Maximal phosphorylation observed). Comparison of phosphorylated S6 levels with IC50 measurements for the mTOR inhibitor In the same cell lines indicate a significant correlation between increased antiproliferative activity of the mTOR inhibitor and increased levels of phosphorylated S6 (e.g. S6 phosphorylation on serines 240 and 244 [using Cell Signaling Technology® antibody catalogue #2215]: n=7, R=−0.746, p=0.00384 by Spearman Rank correlation analysis). A similar correlation was not observed when performing the same analysis with phosphorylated MAPKIERK1/2 (e.g. ERK1/2 phosphorylated on threonine 202 and tyrosine 204 [using Cell Signaling Technology antibody catalogue #9106]; n=7, R=−0.123, p=0.781).

In order to predict sensitivity of e.g. a tumor in a subject to mTOR inhibitors, a similar analysis to that described above is performed using a sample containing tumor tissue from the subject in place of the human tumor cell lines. Phosphorylated S6 levels obtained from the tumor tissue sample may be compared with that obtained from control tissue, or with data obtained from the human tumor cell lines, in order to predict likely responsiveness to an mTOR inhibitor.

1-4. (canceled)

5. A method for determining the sensitivity of a proliferative disease in a subject to treatment with an mTOR inhibitor, comprising determining the level of expression and/or phosphorylation state of S6 in a sample derived from the subject.

6. A method according to claim 5, wherein the proliferative disease comprises a cancer.

7. A method according to claim 5, wherein the mTOR inhibitor comprises rapamycin or a rapamycin derivative.

8. A method according to claim 7, wherein the rapamycin derivative comprises 40-O-(2-hydroxyethyl) rapamycin.

9. A method according to claim 5, comprising determining the level of expression of phosphorylated S6 protein.

10. A method according to claim 5, wherein the sample is derived from a tumor in the subject.

11. A method according to claim 5, wherein increased expression of phosphorylated S6 relative to control is predictive of sensitivity of the proliferative disease to treatment with the mTOR inhibitor.

12. A method of selecting subjects suffering from a proliferative disease for treatment with an mTOR inhibitor, comprising determining the sensitivity of the proliferative disease to treatment with an mTOR inhibitor in each subject by a method as described in claim 5, and selecting those subjects showing increased expression of phosphorylated S6 for treatment with an mTOR inhibitor.

13. A method of treating a proliferative disease in a subject in need thereof, comprising determining the level of expression of phosphorylated S6 in a sample derived from the subject, by a method as described in claim 5, and treating the subject with an mTOR inhibitor if the level of expression of phosphorylated S6 is elevated.
